(19) United States
(12) Patent Application Publication
(10) Pub. No.: US 2014/0220706 A1
(43) Pub. Date: Aug. 7, 2014

(54) Molecularly Imprinted Polymer for Detecting Waterborne Target Molecules and Improving Water Quality

(71) Applicant: Joseph J. Belbruno, Hanover, NH (US)

(72) Inventor: Joseph J. Belbruno, Hanover, NH (US)

(73) Assignee: THE TRUSTEES OF DARTMOUTH COLLEGE, Hanover, NH (US)

(21) Appl. No.: 14/342,125

(22) PCT Filed: Aug. 31, 2012
(86) PCT No.: PCT/US2012/053349

§ 371 Date: Feb. 28, 2014

Related U.S. Application Data
(60) Provisional application No. 61/529,497, filed on Aug. 31, 2011.

(51) Int. Cl. G01N 33/50

(52) U.S. Cl. CPC G01N 33/50
USPC 436/501; 427/58; 427/487; 427/596; 216/13; 427/122

(57) ABSTRACT

This disclosure relates to the field of molecularly imprinted polymers for detecting or removing target molecules.

### RELATED APPLICATIONS

This application claims the benefit of priority of U.S. Provisional Application Ser. No. 61/529,497 filed Aug. 31, 2011, incorporated herein by reference in its entirety.

### BACKGROUND

Molecular imprinting is a technique that is used to produce molecule specific receptors analogous to biological receptor binding sites. Molecular imprinting of a polymer creates a molecularly imprinted polymer (MIP). A MIP is a polymer that is formed in the presence of a target molecule. The target molecule is removed and leaves a complementary cavity behind in the MIP. The MIP formed demonstrates affinity for the original target molecule.

Sensors for most waterborne target molecules are generally active. For example, the sensors require pumps to draw water through a tube. The sensors also require complex analysis after adsorption of the waterborne target molecules, and various extracted components must be separated prior to analysis. Furthermore, the sensors are not specific for a single waterborne target molecule. The sensors are also not real-time, and only provide an indication of toxic levels in a post-exposure mode. Moreover, some waterborne target molecules, such as cyclic volatile methyl siloxanes (cVMS), have been recognized as environmental problems, but there are currently no sensors available for these target molecules.

### SUMMARY

The present disclosure provides embodiments of sensors including an MIP film that provides detection of a target molecules in water, and by providing MIP powders for removal of waterborne target molecules through the use of, for example, a flow cell. The methods involve using the target molecule in the preparation of the MIP films and powders. When the target molecule is removed, it leaves behind an MIP with cavities that are complementary in shape and functionality to the target molecule. The MIP thereby created can bind target molecules in those cavities.

In an embodiment, provided herein is a sensor including a molecularly imprinted polymer (MIP) film for detection of a waterborne target molecule, the MIP film comprising a polymer host for binding the waterborne target molecule. The sensor can be useful in the detection of a chlorinated solvent, such as carbon tetrachloride, organophosphates, cyclic volatile methylsiloxanes, endocrines, endocrine mimics, estrogens, organobromides, and decabromodiphenyl ethers.

In an embodiment, the polymer host of a sensor can be at least one of polyaniline, poly(4-vinylphenol) (P4VP), polyurethane, nylons, poly(2-vinylpyrole), poly(vinylpyridine) (PVP), poly(methylmethacrylate) (PMMA), acrylates, nylon-6, polyethyleneimine, polyurethane, polycarbonate and/or polyvinylpyrrolidinone and polystryrene.

The polymer can be chosen based on its affinity for a target. For example, when the target is an organophosphate, the polymer host can be an acrylate, e.g., PMMA. If the target is a cyclic volatile methylsiloxane, the polymer host can be PMMA, P4VP or PVP.

MIPs disclosed herein can be used for sensors and/or solid phase extraction (SPE). Polymers used to produce the MIPs disclosed herein may be referred to as a polymer host. Molecules disclosed herein for the production of the MIPs can be referred to as a target, a contaminant, or a target molecule.

In an aspect, a sensor for the detection of an waterborne target molecule comprising a molecularly imprinted polymer film and a surface, the molecularly imprinted polymer film comprising a polymer host comprising binding sites for the waterborne target molecule, and the molecularly imprinted polymer film is coated upon the surface. In an embodiment, the sensor has a molecularly imprinted polymer film that is conductive. In an embodiment, the sensor is an electrode. In another embodiment, the sensor is an electrode patterned with an interdigitated grid or circuit. In an embodiment, the sensor is a molecularly imprinted polymer film which is the surface. In another embodiment, the sensor has a molecularly imprinted polymer film that has a thickness equal to or less than about 0.25 inches.

In an aspect, a method for detecting an waterborne target molecule using a molecularly imprinted polymer film or sensor is disclosed whereby the method exposes the molecularly imprinted polymer film coated surface to a solution, measures the resistance to the flow of an electrical current applied to the molecularly imprinted polymer film coated surface, and where the resistance measurement is used to detect the waterborne target molecule in the solution.

In an aspect, a molecularly imprinted polymer film composition is carbon nanotubes coated with a molecularly imprinted polymer layer. In an embodiment, the molecularly imprinted polymer film composition is produced by the method of mixing together a polymer, carbon nanotubes, a target molecule and a first solvent to form a molecularly imprinted polymer solution.

In an aspect, a sensor for the detection of a waterborne target molecule is a molecularly imprinted polymer film and a surface, the molecularly imprinted polymer film is carbon nanotubes coated with a molecular imprinted polymer having binding sites for a waterborne target molecule, and the molecularly imprinted polymer film is coated upon the surface. In an embodiment, the sensor has a molecularly imprinted polymer film that is produced by the method of mixing together a polymer, carbon nanotubes, a target molecule and a first solvent to form a molecularly imprinted polymer solution, and the molecularly imprinted polymer solution coats a surface. In another embodiment, the sensor is a molecularly imprinted polymer solution that coats a surface by electropolymerization, spin casting or laser deposition. In yet another embodiment, the sensor surface is an electrode. In an embodiment, the sensor surface is an electrode patterned with an interdigitated grid or circuit.

In an embodiment, a method for producing a molecularly imprinted polymer film for detection of a waterborne target molecule dissolves a polymer host that is a structural component and/or a conductive component in a first solvent to form a first solution, then adds a target molecule to the first solution, then mixes the target molecule into the first solution to form a molecularly imprinted polymer solution, then coats the molecularly imprinted polymer solution onto a surface and then removes the target molecule to form the molecularly imprinted polymer film. In an embodiment, the method of coating is electropolymerization, spin casting and/or laser deposition. In an embodiment, the method of removing the target molecule includes extracting the target molecule from the molecularly imprinted polymer film using a second solvent, when the polymer host is insoluble in the second solvent, and when the target molecule is soluble in the second solvent. In another embodiment, the method involves a first solvent that has a boiling point lower than the boiling point of the target molecule. In yet another embodiment, the step of removing the target molecule involves evaporating the target molecule from the molecularly imprinted polymer film.

In an embodiment, any one of the above mentioned methods can be used when the target molecule is selected from the group consisting essentially of chlorinated solvents, carbon tetrachloride, organophosphates, cyclic volatile methylsiloxane, endocrines, endocrine mimics, estrogens, organobromides, and decabromodiphenyl ether. In an embodiment, the target molecule is a homolog of the waterborne target molecule. In an embodiment, the first solvent is selected from the group of alcohols, dimethylformamide, and chloroform. In another embodiment, the first solvent is formic acid.

In an embodiment, any one of the above mentioned methods may be used wherein the structural component of the sensor is poly(4-vinylphenol), polyurethane, nylons, poly(2-vinylpyrole), poly(vinylpyridine) (PVP), poly(methylmethacrylate) (PMMA), acrylates, nylon-6, polyethyleneimine, polyurethane, polycarbonate, polyvinylpyrrolidinone and polystryrene. In an embodiment, structural component is polyaniline, carbon nanotubes, and/or single wall carbon nanotubes. In another embodiment, the polymer host is nylon-6 and polyaniline. In yet another embodiment, the polymer host is polyethyleneimine and polyaniline. In an embodiment, the polymer host ranges from about 2 percent to about 15 percent by weight with respect to the first solvent in the first solution. In another embodiment, the target molecule ranges from about 2 percent to about 10 percent by weight with respect to the first solvent in the molecularly imprinted polymer solution. In yet another embodiment, the molecularly imprinted polymer solution comprises from about 2 to about 15 percent by weight of the component and the structural component and also is from about 2 to about 10 percent by weight of the target molecule. In an embodiment, the molecularly imprinted polymer solution is from about 2 to about 15 percent by weight of polyaniline and polyethyleneimine and from about 2 to about 10 percent by weight of the target molecule. In an embodiment, the molecularly imprinted polymer solution is from about 2 to about 15 percent by weight of polyaniline and polyethyleneimine; and from about 2 to about 10 percent by weight of formic acid. In another embodiment, the molecularly imprinted polymer film composition has a molar ratio of about 1 to 1 of the component and the structural component. In yet another embodiment, the molecularly imprinted polymer film composition has a molar ratio of about 1 to 1 of the conductive component and the structural component, and the target molecule is selected from the group of chlorinated solvents, carbon tetrachloride, organophosphates, cyclic volatile methylsiloxane, endocrines, endocrine mimics, estrogens, organobromides, and decabromodiphenyl ether. In an embodiment, the molecularly imprinted polymer film composition has a molar ratio of about 1 to 1 of polyaniline to polyethyleneimine, and the target molecule is carbon tetrachloride.

In an embodiment, a molecularly imprinted polymer film is disclosed for the detection of a waterborne target molecule produced by any one of the above mentioned methods.

In an aspect, a method for removing a waterborne target molecule from a solution involves using a molecularly imprinted polymer film for the detection of a waterborne target molecule produced by any one of the above mentioned methods using a chromatographic process in which the solution is passed through the molecularly imprinted polymer film.

In another aspect, a solid phase extraction molecularly imprinted polymer is a polymer host that has binding sites for a waterborne target molecule. In an embodiment, the solid phase extraction molecularly imprinted polymer is a polymer host comprising a structural component, a conductive component and a target molecule. In another embodiment, the solid phase extraction molecularly imprinted polymer structural component is poly(4-vinylphenol), polyurethane, nylons, poly(2-vinylpyrole), poly(vinylpyridine) (PVP), poly(methylmethacrylate) (PMMA), acrylates, nylon-6, polyethyleneimine, polyurethane, polycarbonate, polyvinylpyrrolidinone and/or polystryrene. In yet another embodiment, the solid phase extraction molecularly imprinted has a conductive component that is polyaniline, carbon nanotubes, and/or single wall carbon nanotubes. In another embodiment, the solid phase extraction molecularly imprinted polymer has a polymer host that is nylon-6 and polyaniline. In yet another embodiment, the solid phase extraction molecularly imprinted polymer has a polymer host that is polyethyleneimine and polyaniline. In an embodiment, the solid phase extraction molecularly imprinted polymer has a polymer host from about 2 percent to about 15 percent by weight and is also from about 2 to about 10 percent by weight of the target molecule. In an embodiment, the solid phase extraction molecularly imprinted polymer has a molar ratio of about 1 to 1 of the component and the structural component, and the target molecule is selected from the group of chlorinated solvents, carbon tetrachloride, organophosphates, cyclic volatile methylsiloxane, endocrines, endocrine mimics, estrogens, organobromides, and decabromodiphenyl ether. In yet another embodiment, the solid phase extraction molecularly imprinted polymer is a waterborne target molecule that is selected from the group of chlorinated solvents, carbon tetrachloride, organophosphates, cyclic volatile methylsiloxane, endocrines, endocrine mimics, estrogens, organobromides, and decabromodiphenyl ether. In an embodiment, the solid phase extraction molecularly imprinted polymer is a powder form of a MIP film produced by any one of the above methods.

In yet another embodiment, a method for removing a waterborne target molecule from a solution uses a solid phase extraction molecularly imprinted polymer in a chromatographic process in which a solution is passed through the solid phase extraction molecularly imprinted polymer.

Embodiments of the sensors provided for herein allow for the detection of even a single kind of waterborne target molecule. The disclosure provides methods to produce a sensor including a conductive MIP film and/or MIP film. The methods involve using the target molecule in the preparation of the MIP films and sensors comprising MIP films.

Additional embodiments and features are set forth in part in the description that follows, and in part will become apparent to those skilled in the art upon examination of the specification. A further understanding of the nature and advantages of the present invention may be realized by reference to the remaining portions of the specification and the drawings, which forms a part of this disclosure.

### DESCRIPTION OF THE DRAWINGS

FIG. 1 illustrates a simplified molecularly imprinted polymer solution in an embodiment.

FIG. 2 is a flow chart illustrating the steps of a modified phase inversion process for producing MIPs, in an embodiment.

FIG. 3A illustrates an exemplary test strip in an embodiment.

FIG. 3B illustrates an exemplary test strip with water spray containing color reagents in an embodiment.

FIG. 3C illustrates an exemplary test strip in a vial with liquid color reagents in an embodiment.

FIG. 3D illustrates an exemplary test strip with color reagents covalently bonded to the MIP film in an embodiment.

FIG. 4 illustrates an exemplary multi-band test strip in an embodiment.

FIG. 5 illustrates an exemplary patch tester in an embodiment.

FIG. 6 illustrates an exemplary conductive sensor including an MIP film in an embodiment.

FIG. 7 illustrates an embodiment of a dip-stick tester.

### DETAILED DESCRIPTION

The present disclosure may be understood by reference to the following detailed description, taken in conjunction with the drawings as described below. It is noted that, for purposes of illustrative clarity, certain elements in the drawings may not be drawn to scale.

### MIP Films and Sensors

The present disclosure provides methods for producing MIPs. The polymer of a MIP contains binding sites for the target molecule. Without being bound by theory, the target molecule binds to the binding sites in the polymer layer via physical or chemical forces such as electrostatic interactions, Van der Waals forces, ionic bonds or even covalent bonds. The polymer layer of the MIP may also be referred to as the polymer host. The polymer layer (polymer host) of the MIP can contain a structural polymer component (structural component) and a conductive polymer component (conductive component). The structural component of the polymer layer provides the structural support for the polymer layer of the MIP. In an embodiment, the structural component primarily forms the binding site of the polymer host. In an embodiment, the conductive component of the polymer host is a conductor of electrons and allows for the flow of an electrical current through the polymer host.

In an embodiment, the physical property associated with the presence of a target molecule in a MIP film is a change in the resistance of the MIP film with or without the target molecule bound. As used herein, a film generally refers to a coating of a surface. An embodiment of a film is coating of a surface by a polymer or MIP. In one embodiment a MIP film is from about 1 Å to about 10,000 Å. In general, MIP film sensor functionality depends upon detecting differences in the resistivity of the MIP film as a function of the adsorption of a target molecule. In an embodiment, MIP film sensors can be tested for their ability to detect waterborne target molecules by using various flow through chambers or otherwise exposing the MIP film sensors disclosed herein to a sample of a solution, such as a liquid.

In an embodiment, the resistance, R, of the MIP films is measured with a multimeter when a constant current is being applied using two contacts to the MIP films and/or sensors.

The conductive polymer component of the polymer host provides a conductive path for the flow of current within the polymer host. In an embodiment, the polymer host consists of only a conductive component, or only a structural component. In another embodiment, the polymer host consists of any percent composition of both the structural component and the conductive component.

The present disclosure provides methods for producing molecularly imprinted polymers (MIPs). Potential candidates for MIP polymers are those polymers that chemically interact with a target molecule, or interact with polar molecules so that the MIP-molecule interaction would be electrostatic. These MIP polymers (alternatively referred to as polymer hosts) include, but are not limited to, polyaniline, poly(amino acids), poly(4-vinylphenol) (P4VP), polyurethane (PU), nylons, poly(2-vinylpyrole) (PVPy), poly(vinylpyridine) (PVP), poly(methylmethacrylate) (PMMA) and polystyrene (PS). This list includes polar and non-polar materials. Depending upon the MIP polymer of choice, the solvents in which the MIPs have high solubility can include, but are not limited to, alcohols, dimethylformamide, formic acid and chloroform.

The present disclosure provides embodiments of MIP films and sensors for the detection and/or measurement of target molecules in water samples, solutions and/or colloidal suspensions, for example. In this disclosure, a polymer host includes a structural component for a target molecule that is present during the formation of the MIP. In an embodiment, polyurethane is a structural component of a polymer host of an MIP. The polymer host can also include a conductive component, such as polyaniline.

In an embodiment, the target molecules are in a solution, such as a homogeneous solution, heterogeneous solution, or colloid solution for example. In an embodiment, target molecules can include chlorinated solvents, such as carbon tetrachloride. In an embodiment, the polymer host can be PMMA for shape recognition, and/or P4VP or PVP for electrostatic recognition, or both. In an embodiment, target molecules can also include organophosphates. In an embodiment, organophosphate target molecules can be components of pesticides and weaponized nerve agents. For such target molecules, the polymer host can be PMMA and/or acrylate. Target molecules can also include cyclic volatile methylsiloxanes, which are a major component of personal care products, and have recently been found in water samples throughout the developed world.

In an embodiment, the polymer host of MIP films and sensors for cyclic volatile methylsiloxanes target molecules can be PS, PMMA and/or PU. The target molecules can also include endocrine molecules, such as estrogens, as well as mimics thereof. In an embodiment, the polymer host of MIP films and sensors for endocrine molecules, such as estrogens, as well as mimics thereof, can be PS. Additional target molecules detected by MIP films and sensors disclosed herein can further include flame retardants, with organobromides such as decabromodiphenyl ether, and organophosphates. In an embodiment, the polymer host of MIP films and sensors for flame retardants, with organobromides such as decabromodiphenyl ether, and organophosphates, can be PMMA, related acrylates, P4VP and/or PVP.

In an embodiment, MIP films can be regenerated by extracting and/or evaporating target molecules from a MIP film by soaking or washing in a solvent in which the polymer host is insoluble, but the target molecule is soluble. In an embodiment, the target molecules can be removed from the MIP binding sites through extraction and/or evaporation processes. The MIP films are then washed and dried to allow the solvent and the target molecule to be separated from the MIP films. After extraction and/or evaporation of the target molecule, the MIP films are ready to detect target molecule again.

Sensing using conductive polymer films can be performed either by coating the surface of an electrode with the doped polymer, a MIP containing bound target molecule, and measuring the cell potential with reference to a redox electrode, or by making a true planar, chemiresistive structure. The latter can be used with a variety of conductive polymers or composites, and may be designed to create higher values of resistance (signal).

MIP film based sensors provide rapid detection of target molecules. The MIP film based sensors disclosed herein can be planar structures designed for a quick time response through choosing an appropriate geometry and materials for the MIP film based sensor. In some embodiments provided herein, a spin casting method for preparing thin MIP films on lithographically produced electrodes that detect target molecules in a solution are disclosed.

Conductivity measurements of embodiments of the sensors presented herein are indicative of the binding of target molecules. In an embodiment of the MIP films and MIP film based sensors disclosed herein, data are reported as normalized resistance (or the change in resistance), referenced to an initial or background value. The change in the resistance value, and the rate of change in the resistance (the slope), are proportional to the quantity and identity of the molecule adsorbed. The change in the resistance value, and/or the rate of change in the resistance may be used to quantify and/or detect the target molecule. Additional evidence of target molecules being bound in a MIP film can be obtained through IR spectroscopy and gas chromatographic experiments.

The morphology of MIP films disclosed herein can be further characterized by scanning electron microscopy.

One of the benefits of the methods disclosed herein over conventional methods for detection of the waterborne target molecules is the specificity of the MIP films for target molecules. In an embodiment, waterborne target molecules can be adsorbed by a MIP film passively. There is no need for the use of a pump or other moving parts for actively drawing a solution into the MIP film sensing device. Moreover, the device can provide real-time indications of exposure levels and the device is small enough for a user to wear. It will be appreciated by those skilled in the art that configuration, shape, and dimensions of the sensor can vary for particular applications.

### Methods of Making MIP Films and Sensors

The present disclosure provides methods for making MIPs and sensors that use MIPs. In an embodiment, MIPs are made by mixing together a structural component, a conductive component, a target molecule and a first solvent. In an embodiment, a structural component is a structural polymer. In an embodiment, a conductive component is a conductive polymer. In an embodiment, the solution of the polymer components, the first solvent, and the target molecule is a molecularly imprinted polymer solution. The molecularly imprinted polymer solution can then be coated onto a surface such as an electrode and allowed to dry. When the molecularly imprinted polymer solution is drying, the polymers form the binding sites for the dissolved target molecules as the polymer layer polymerizes around the target molecules. Next, the target molecule is selectively removed from the MIP layer by either evaporation of the target molecule or through extraction with a solvent that selectively dissolves the target molecule, but does not dissolve the polymer host.

The solvent used in making the MIPs can boil at a lower temperature than the target molecule. This allows the target to form recognition sites during spin or dip coating. An organic solvent can then be used to remove the target. The organic solvent should be incompatible with the polymer host to promote precipitation of the MIP. Alternatively, the volatile organic molecule or target can be evaporated from the MIP if the solvent has a lower boiling point than the target.

A polymer host of a MIP film based sensor can contain both structural components, such as PEI as well as conductive components such as PANi. The structural component of the MIP film is useful for forming the support structure of the pockets where the target molecules bind. The conductive component of the MIP film is useful for allowing an electrical current to flow through the polymer host. The resistance to the flow of the electrical current changes depending upon whether or not the binding sites in the MIP are bound with target molecules. Certain non-limiting embodiments of the MIP sensors provided for herein have polymer hosts containing polyaniline conductive components incorporated into polyethyleneimine structural components as thin PANi/PEI composite films prepared by spin-casting for waterborne target molecule detection via changes in conductivity. In an embodiment, the sensors have significant increases in the resistance of the MIP films upon exposure to target molecules in a liquid solution. The films are responsive to other volatile organic vapors, but at significantly reduced levels. The morphology of various embodiments of the MIP films have a porous surface well-suited to liquid phase adsorption.

There are various techniques for depositing films including electropolymerization, spin casting and laser deposition. In certain embodiments of the present disclosure, PANi is employed to directly measure the target concentration in concert with a second polymer included in composite materials to improve the porosity of the film.

In an embodiment, MIP films are spin-cast composites of PANi and PEI. PANi in its conductive form is insoluble, but the emeraldine base may be dissolved in several solvents in which PEI is also soluble. In an embodiment, the spin casting solution can be produced as a 5 percent (by weight) solution in each of the two polymers, structural component and conductive component. In an embodiment, molecularly imprinted polymer solutions can be coated onto surfaces by electropolymerization, spin casting or laser deposition.

In an embodiment, a PANi/PEI polymer layer can be spin-coated onto an electrode. An aliquot of molecularly imprinted polymer solution is dropped onto the electrodes and allowed to spread. The spin-coater device spins the electrode at a given rpm for an amount of time resulting in the deposition of films. In an embodiment, the thickness of the MIP films is about 300 nm.

In an embodiment MIP film sensors are constructed on oxidized silicon substrates with a PANi/PEI composite film as the active element above the electrode. In one non-limiting embodiment, prime grade silicon wafers with a thermally deposited oxide layer are used for the substrate. These oxide layers can be patterned by photolithography and subsequently wet etched to produce electrodes, which are then subjected to a vapor deposition of chromium or other metals and an overlayer of nickel or other like metals. Lift off can be accomplished using acetone, with final rinses of water to produce an electrode patterned into an interdigitated grid.

### Embodiments of MIP Films and Sensors

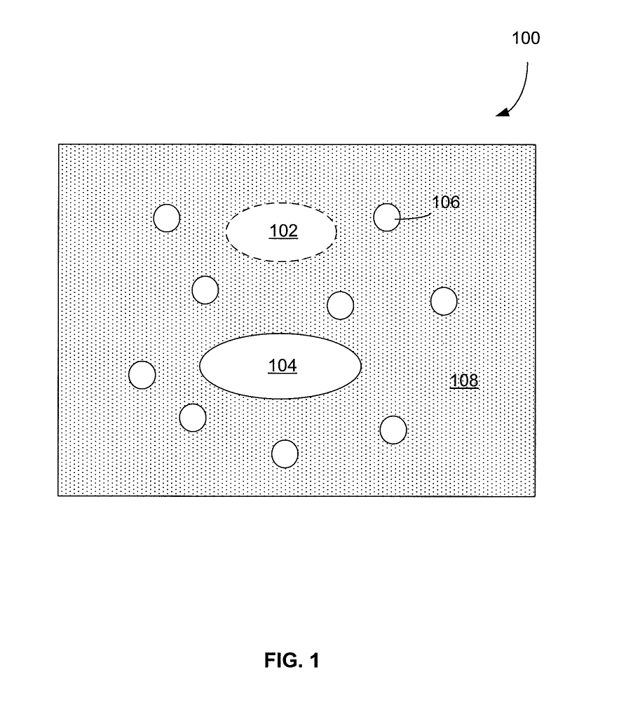

FIG. 1 illustrates an embodiment of a simplified molecularly imprinted polymer solution. Molecularly imprinted polymer solution 100 includes a chemical component 102 dissolved in a solvent 108 and a structural component 104, also dissolved in the solvent 108. Polymer solution 100 also includes target molecule 106 dissolved in solvent 108. As illustrated in FIG. 1, target molecule 106 is bonded to the chemical component 102 in the polymer solution 100, also referred to a MIP solution.

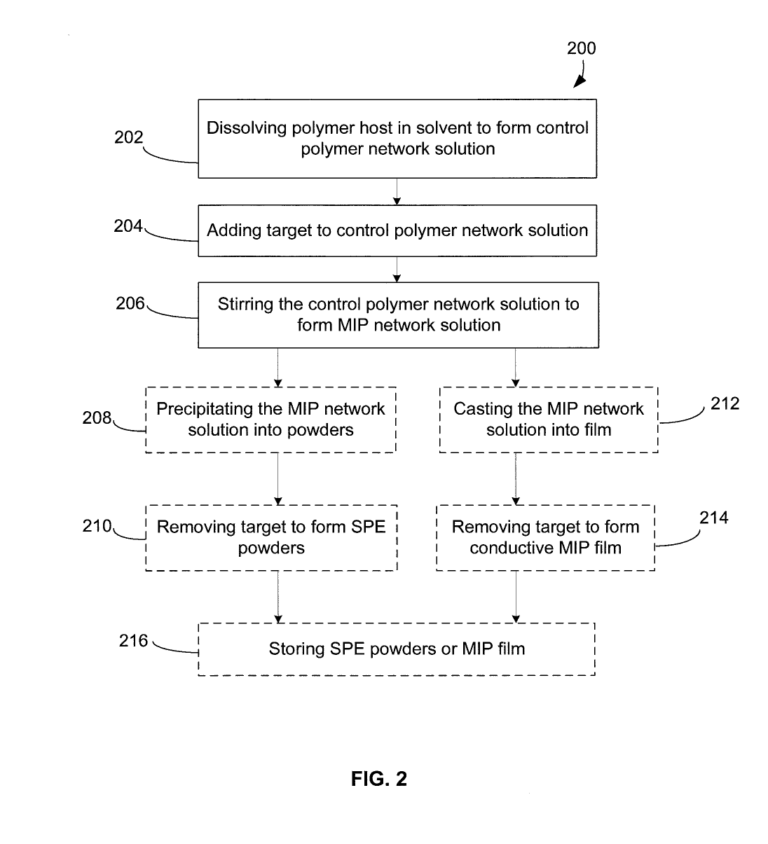

In an embodiment, conductive MIPs are produced by a modified phase inversion process, as illustrated in FIG. 2. A polymer host generally includes a conductive component and a structural component for a target molecule that is present during the formation of the molecularly imprinted polymer (MIP). For example, polyaniline is a conductive polymer of the host, and nylon-6, polyethyleneimine, or polyvinylpyrrolidinone may be a structural component of the polymer host when these two polymers are used simultaneously. In an embodiment, the polymer host is a conductive polymer. In one embodiment, the polymer host is a structural polymer. In another embodiment, the MIP contains only a structural component such as polyvinylpyrrolidinone and is cast or otherwise coated upon a conductive component or surface such as carbon nanotubes. In an embodiment, the polymer host is only a conductive polymer.

In an embodiment, a process for making MIP films of the present disclosure 200, also referred to as a modified phase inversion process 200, includes dissolving the polymer(s), e.g., polyaniline and nylon-6, of the polymer host sequentially in a suitable solvent to form a first solution at step 202. After dissolution of the polymer host in the solvent, the target molecule (e.g., waterborne target molecule) or a molecule with similar size and chemical properties as the target molecule is added to the first solution at step 204. In an embodiment, the process 200 also includes stirring the first solution to insert or otherwise incorporate the target molecule into the polymer host to form an MIP polymer solution at step 206.

In an embodiment, process 200 further includes precipitating the MIP solution into powders at step 208 and removing the target molecule by addition of a solvent. A suitable solvent for removal or extraction of the target molecule from the MIP is one in which the polymer host is poorly soluble in, but one in which the target molecule is soluble to very soluble in. Using the selective solubility of the target molecule over the polymer host allows for the MIP film to act as a SPE because the target molecule may be selectively bound and then extracted from the polymer host. In an embodiment, the process form making the MIP films disclosed herein can be used to produce SPE powders at step 210. After drying, the SPE powders are ready for use in a solid phase extraction (SPE) tube. In an embodiment, process 200 further includes storing the SPE powders or MIP film at step 216.

In an embodiment, SPE powders are useful for purifying a water based solution by selectively binding a waterborne target molecule. In an embodiment, SPE powders can act as a stationary layer in a purification process using column based chromatography through which a water based solution is filtered or flows through. In another embodiment, SPE powders can be molded, sintered or otherwise formed into films, layers, and/or structures useful as stationary layer, frit or other matrix within a flow through column. In a non-limiting embodiment, SPE MIP films and/or structures can be used in various chromatographic purification techniques such as reversed-phase chromatography, ion exchange chromatography, affinity chromatography, liquid chromatography (including high performance liquid chromatography), displacement chromatography, planar chromatography and column chromatography. In one embodiment, a water solution can be purified by passing through a flow through column made from SPE powders that contain MIP films that selectively bind waterborne target molecules. In an embodiment, water based solutions can include streams, lakes, ground water, or any body of water containing or potentially containing a waterborne target molecule.

In another embodiment, process 200 includes casting the MIP solution into a film at step 212. The MIP film may or may not contain the target molecule. In one embodiment, the cast MIP film does not contain the target molecule at step 214. The MIP film can be used as a membrane or as a sensor and can be formed via any number of techniques, such as spin coating, drop casting, ink jet printing or dip coating, among others. A spin coating procedure for an MIP film is described in the US patent publication US 2010/0039124 A1, entitled “Molecularly Imprinted Polymer Sensor Systems And Related Methods,” filed on Jun. 14, 2007, which is incorporated herein by reference. After drying, the MIP film is ready for use in a film based sensor. In an embodiment, process 200 produces a thin film MIP that can serve as part of a sensing device to detect waterborne target molecule s.

The interaction between a polymer host and a target molecule in a MIP can involve non-covalent bonding, such as hydrogen bonding, between the polymer host and the target molecule. The binding interaction can exploit other electrostatic forces in conjunction with shape recognition, but the interaction between polymer host and the target molecule is not limited to non-covalent forces and can also include ionic and/or covalent chemical bonds between the target molecule and the polymer host.

When the target molecule is removed via extraction or evaporation or by other removal means, it leaves behind a MIP with cavities that are complementary in shape to the target molecule and act as a binding site to the target molecule or similar molecules. The MIP films disclosed herein are capable of rebinding target molecules through subsequent rounds of use when the MIP is regenerated between measurements by removing the target molecule from the MIP before the next use of the MIP film and/or sensor.

In another embodiment, MIPs can be produced by dissolving the polymer or polymer host components, i.e., conductive and structural, and target molecules in a first solvent to form a molecularly imprinted polymer solution. In one embodiment, the target molecule forms between about 1 and about 30 weight percent of the molecularly imprinted polymer solution. In a preferred embodiment, the target molecule forms between about 2 and about 20 weight percent of the molecularly imprinted polymer solution. In a more preferred embodiment, the target molecule forms between about 2 and about 15 weight percent of the molecularly imprinted polymer solution.

In an embodiment of a MIP of the present disclosure, the molecularly imprinted polymer solution has a molar ratio of from about 10:1 to about 1:1 to about 1:10 of the structural component to the conductive component. In an embodiment, the molecularly imprinted polymer solution is from about 1 to about 30 percent of the target molecule or homolog by weight. In a preferred embodiment of a MIP of the present disclosure, the molecularly imprinted polymer solution has a molar ratio of from about 5:1 to about 1:1 to about 1:5 of the structural component to the conductive component. In a preferred embodiment, the molecularly imprinted polymer solution is from about 2 to about 20 percent of the target molecule or homolog by weight. In a more preferred embodiment of a MIP of the present disclosure, the molecularly imprinted polymer solution has a molar ratio of from about 1:1 of the structural component to the conductive component. In a more preferred embodiment, the molecularly imprinted polymer solution is from about 2.5 to about 10 percent of the target molecule or homolog by weight.

In an embodiment of a MIP of the present disclosure, nylon-6 is used as the structural component and polyaniline is used as the conductive component for the polymer host of a MIP film having waterborne target molecule as the target molecule. In an embodiment, homologs of the waterborne target molecule can be used in the production of a MIP film useful for the detection of waterborne target molecules as the target molecule. In some embodiments, a first solvent can be used as both a solvent for dissolving the structural and conductive components as well as dissolving a waterborne target molecule or homolog thereof.

The first solvent should be suitable for each component of the polymer host and the target molecule. For example, polyaniline, nylon and a waterborne target molecule are all soluble in a first solvent. The polymer hosts and solvents can vary for a particular target molecule of interest. Non-limiting examples of solvents can include alcohols, dimethylformamide, water, formic acid and chloroform.

In an embodiment, after dissolving the polymer host components, 2 to 10 weight percent of the target molecule is added in the polymer solution, followed by stirring for about 20 hours to uniformly mix the target in the polymer solution and form the molecularly imprinted polymer solution. In general, when a higher target concentration is used, the sensitivity of the MIP to target detection increases. However, the MIP's detection or separation for a particular molecule or molecular specificity is reduced.

In an embodiment, thin films are produced by spin casting onto glass substrates at a spin rate of about 4000 rpm for a period of about 30 seconds and allowed to air dry for about 1 hour. The final film can be stored until needed for use to rebind the target.

The MIP films produced in process 200 are suitable for use as a sensor that reports the presence of the target molecule via, for example, a color change, either through a polymer incorporated chromaphore or an externally added reagent. Such a film can be built into a capacitor to monitor dielectric changes due to the presence/absence of the target molecule. Alternatively, if the polymer is conductive, a resistor that monitors the presence of the target molecule via conductivity changes can be constructed. Conductivity can be incorporated into the MIP by using a conductive polymer such as polyaniline and a structural polymer component that provides the actual recognition sites.

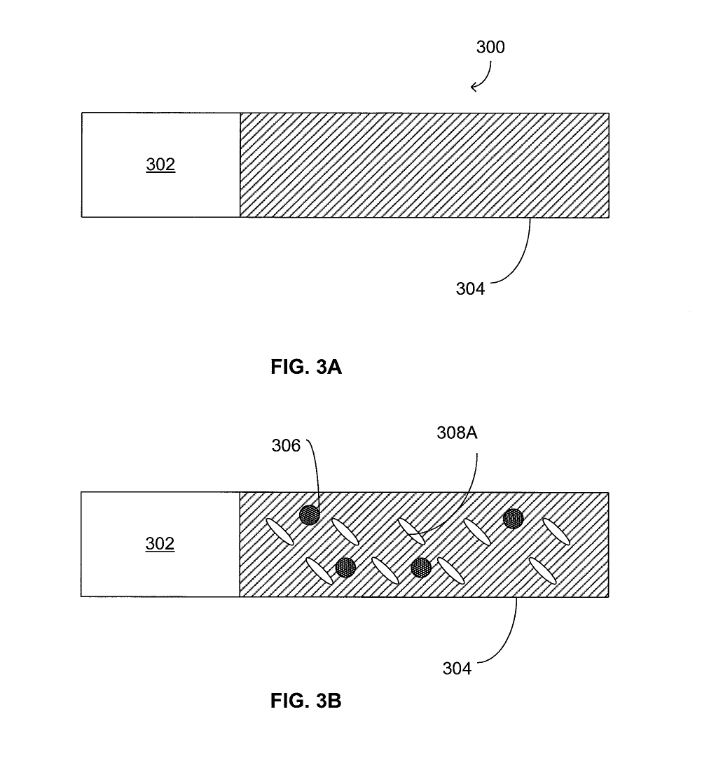

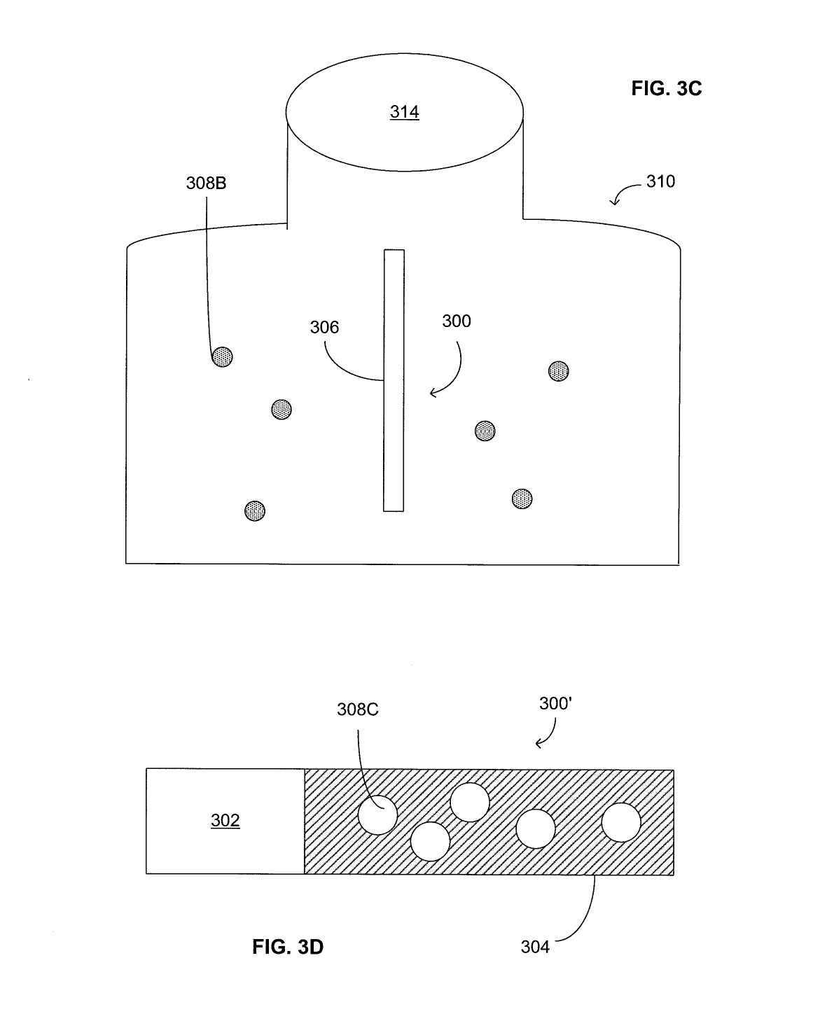

There are various techniques for visual identification or electrical detection of MIPs exposed to their target molecules. These techniques can use static adsorption, flow absorption or capillary action. FIG. 3A illustrates an exemplary test strip 300 that includes a plastic substrate 302. A portion of the plastic substrate 302 is covered with an MIP film 304. FIG. 3B illustrates that a sample solution 306 can be deposited on MIP film 304 and followed by washing sample solution 306 with a water spray containing a color reagent 308A. When a target molecule binds to the color reagent, the test strip changes color to indicate a “Yes” for the presence of the target. Otherwise, if no target molecule binds to the color reagent, there is no color change, which indicates “No” for the presence of the target. Color reagent 308A can also provide a range of concentration of the target based upon color intensity.

Alternatively, instead of using a water spray containing color reagent 308A, test strip 300 can be used in a vial 310 with a liquid color reagent 308B, as illustrated in FIG. 3C. In an embodiment, a user can open cap 314 of vial 310, apply sample solution 306 to the MIP film 304, wash off any excess sample, and deposit test strip 300 in vial 310, followed by sealing cap 314 and shaking vial 310 to monitor color change of color reagent 308B.

FIG. 3D illustrates test strip 300′ with color reagent 308C covalently bonded to the MIP film. Color reagent 308C is also capable of covalently bonding with a target molecule. If target sample 306 is present on the MIP film 304, color reagent 308C will change its color to indicate the detection of the target sample.

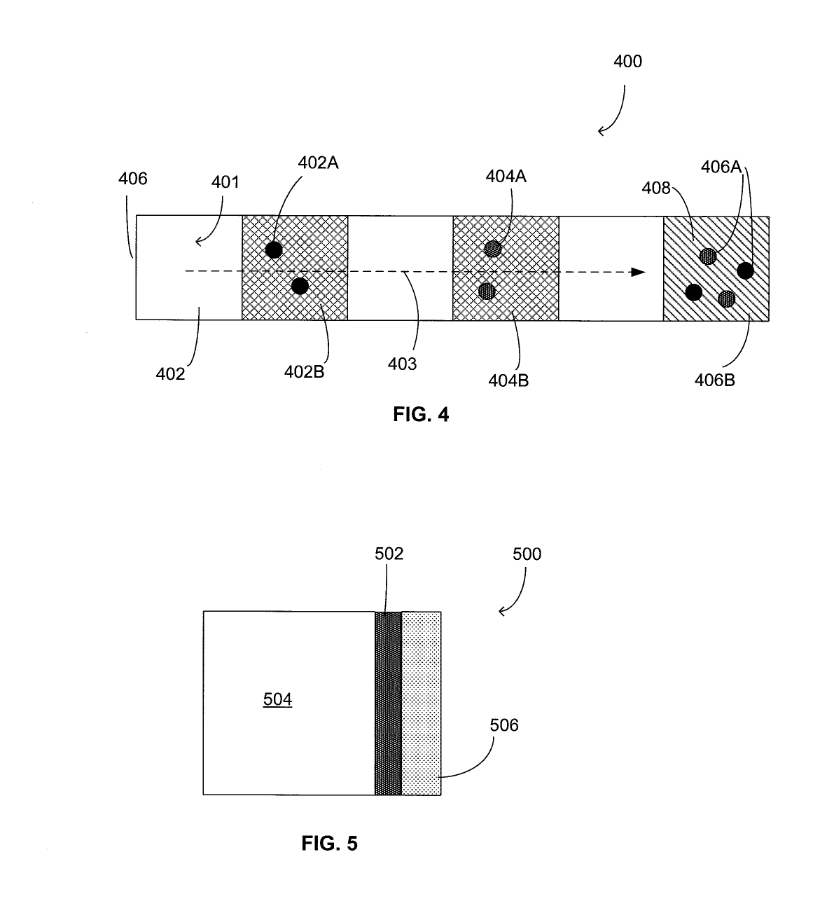

FIG. 4 illustrates an exemplary multi-band test strip. Multi-band test strip 400 includes a plastic substrate 402 covered with an adsorbing layer 401 (e.g., a paper layer, such as utilized in paper chromatography strips or membranes such as those used in lateral flow assays). Multi-band test strip 400 is useful when reagents must be added sequentially. A liquid sample can be added at end 406 to flow through reagent bands 402B and 404B, in the direction of arrow 403. The liquid sample flow picks up reagents 402A and 404A in reagent bands 402B and 404B respectively. A final reagent band 406B includes both a reagent 406A and an MIP film 408. Upon reaching reagent band 406B, if the target is present and has reacted with reagents 402A and 404A, it will react with MIP film 408, and will provide a color change to indicate the presence of the target. Otherwise, no color change occurs.

FIG. 5 illustrates a cross-sectional view of an exemplary sensor for a target molecule. The sensor 500 includes a thin, easily broken membrane 502 that is sandwiched between a reagent reservoir 504 and an MIP film 506. A sample can be applied to the MIP, and excess sample can be washed off. Sensor 500 can be twisted so that the membrane 502 breaks and the reagents from reservoir 504 flow into the MIP film 506 and react with the target to provide color to indicate the presence of the target in the sample. Otherwise, when there is no color, sensor 500 indicates that the sample does not contain the target.

All of these diagnostic methods can be “Yes” or “No” tests for the presence of the target or one can use visual comparisons of the color intensity or a small meter to quantitatively measure the concentration of the target.

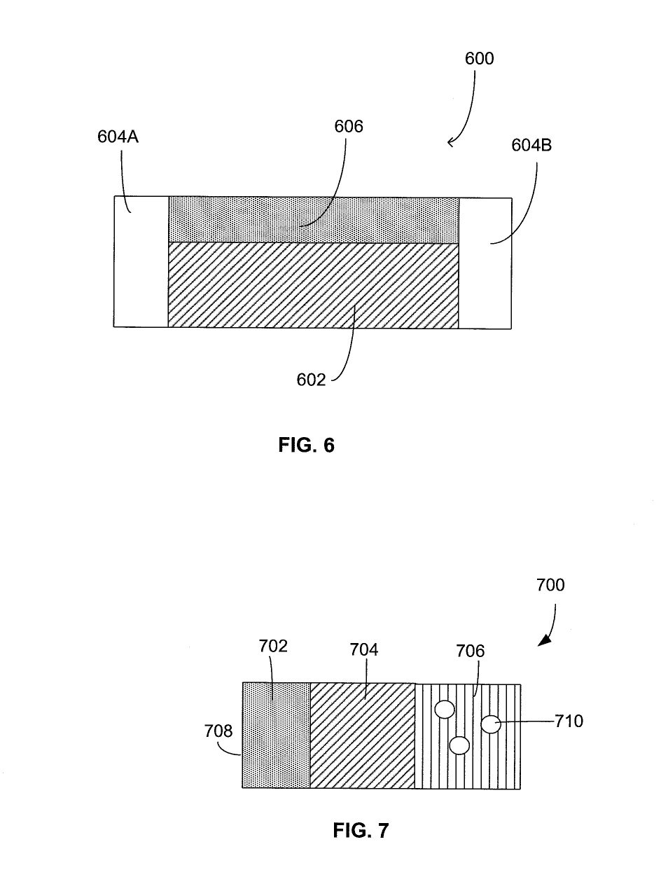

FIG. 6 illustrates a conductive sensor. The sensor 600 includes two electrodes 604A and 604B with a MIP film 606 between the electrodes. MIP film 606 is supported by a substrate 602 between the electrodes. The substrate 602 is an insulator, for example, a plastic or a glass. There are many other possible configurations for the conductive sensor.

The MIP film can be deposited between the electrodes 604A-604B. A small electric current flows through the MIP film 606, so that the resistance of the MIP film 606 can be measured. In an embodiment, MIP film 606 is conductive. For example, MIP film 606 can include a conductive polymer, such as polyaniline.

In an embodiment, a target waterborne molecule may be electronically monitored as a function of time as a water sample flows through a flow cell containing sensor 600. This method of monitoring a target accumulates data for a particular target molecule in real time. In an embodiment, radio frequency identification (RFID) technology can be applied to these sensing systems such that the concentrations of the waterborne target molecules can be reported in real-time.

In one embodiment, a single MIP film sensing device incorporates a large number of sensors for a range of target molecules, so that simultaneous measurements of all targets are obtained with a single sample. Non-limiting embodiments of waterborne target molecules include heavy metals and their ions.

In an embodiment, the MIP film can also be formed from MIP-coated carbon nanotubes (CNTs) and/or single wall carbon nanotubes (SWNTs). The tennis CNT and SWNT as used herein are generally interchangeable with SWNTs being a kind of CNT. The MIP-coated CNTs can be used when it is difficult to find a conductive polymer host for a particular target. The MIP-coated CNTs can also be used when it is desirable to have more uniformly sized MIP powders for follow-up analysis by techniques such as HPLC.

MIP films disclosed herein are useful as personal sensors for detecting exposure to harmful target molecules. The sensors that can be worn by a user in contact with a solution that could be contaminated by target molecules.

In an embodiment, a MIP film personal sensor, a MIP film, and a MIP film sensor may employ RFID technology to report values for exposure to the target molecule in real time.

An MIP based sensor can also be an electronic sensor with an adsorbent tip that is dipped into a water sample or other solution which flows into an MIP film between two electrodes of a sensor, such that electrical conductance of the solution is measured between the two electrodes. The electrical conductance measured varies with the amount of target molecule that is retained or bound in the MIP film of the electronic sensor. The electronic sensor can also be constructed so that the sensor is in the form of a typical commercial tester, such as a pregnancy testing kit, in which a sample is deposited on the sensor and capillary action transports the sample to a MIP film detection and/or measurement zone of the sensor.

A sensor including a MIP for detection of a single target molecule can be a “dip-stick” that is inserted into a water sample and develops a color that is dependent upon the quantity of target adsorbed by the MIP. FIG. 7 illustrates an exemplary dip-stick tester that includes a dip section 702 at one end, and MIP-coated nanoparticles or microparticles 704 in contact with dip section 702. Dip-stick tester 700 can also include an MIP film 706 at an opposite end from dip section 702. MIP film 706 can have color reagents bonded thereto. A sample 708 is added to dip section 702, and travels along dip-stick tester 700 from dip section 702 to the MIP-coated particles 704. If a target is present in sample 708, the target binds to MIP-coated particles 704. When particles 704 with the target reach MIP film 706, the target binds MIP film 706 and does not continue to travel along dip-stick tester 700. In a particular embodiment, for a positive test, no color develops, while color develops for a negative test. It will be appreciated by those skilled in the art that the dip-stick tester can vary in color and configuration. For example, MIP-coated particles 704 and MIP film 706 can be combined into a single MIP film that is formed from a mixture of polymer host, target, and CNTs, and followed by removal of the target.

### Carbon Nanotube MIP Sensor

In a non-limiting embodiment, carbon nanotube sensors coated with a MIP can be used to measure and/or detect waterborne target molecules. Resistivity measurements of embodiments of sensors with MIP coated carbon nanotubes with and without target molecules bound could demonstrate the detection of target molecules by these MIP coated carbon nanotube sensors.

In an embodiment, MIP coated carbon nanotube films can be cast or otherwise coated upon surfaces to create target molecule specific sensors. In general, a target molecule can be dissolved in a first solvent along with a host polymer that is non-conductive to make a structural component only MIP solution. The structural component only MIP solution can then be mixed with a solution containing carbon nanotubes. The MIP and carbon nanotube solution can then be cast upon a surface, such as an electrode, forming a MIP coated carbon nanotube film on a surface.

### EXAMPLES

### Preparation of PANi/PEI Composite Solutions

In a prophetic example, poly(aniline) is purchased from Polysciences, Inc. as the undoped, emeraldine base form with a molecular weight of 15,000 and a conductivity of 10e-¹⁰ S/cm. Branched poly(ethyleneimine), PEI, with a molecular weight 70,000 g/mol would be obtained from Alfa-Aesar as a 30% aqueous solution. Formic acid, >98%, was purchased from EMD Chemicals and used to dissolve the polymers prior to spin casting. Waterborne target molecule would be purchased from Fisher Scientific as formalin solution (37% waterborne target molecule) containing both water and a small quantity of methanol. All reagents can be used as received without any further treatment.

The polymer films for detecting waterborne target molecules can be spin-cast composites of PANi and PEI. PANi in its conductive form is insoluble. However, the emeraldine base may be dissolved in several solvents. A spin casting solution of PANi/PEI can be produced as a 5% (by weight) solution in each of the two polymers. As a result of the inclusion of doped-PANi, protonated solutions are green, while solutions of the unprotonated material are deep blue.

### Construction of Conductive Devices

In another prophetic example, conductive sensors are constructed on oxidized silicon substrates with the PANi/PEI composite film as the active element above the electrode. In one prophetic example, prime grade silicon wafers with a 5000 Å thermally deposited oxide layer are used for a substrate. The films could be patterned by photolithography and subsequently wet etched to produce the final electrodes, for example with a total area of 376 mm², and could also have a vapor deposition of 1000 Å of chromium and the 200 Å overlayer of nickel. Lift off could be accomplished using acetone, with final rinses of water. The resulting electrode would then be patterned into an interdigitated grid with 40 μm fingers and 20 μm spacing.

In a further step of a prophetic example for the construction of conductive devices, a polymer layer would be spin-coated onto the electrode by using an aliquot of 1 mL of solution that would be dropped onto the electrodes and allowed to spread for 20 seconds. The spin-coater would then be brought up to 1800 rpm for 30 seconds. The resulting deposition of films could have a thickness of about 300 nm.

Background resistance values would then be measured, and the sensor would be ready for use in binding studies. The morphology of the thin films could be further investigated by scanning electron microscopy using a FEI Company, XL-30 ESEM-FEG field emission gun environmental scanning electron microscope.

### Sensor Response

The physical property associated with presence of the target molecule in the film is the change in the resistance. Sensor functionality depends upon detecting differences in this property as a function of the adsorption of the target waterborne target molecule onto the device.

The resistance, R, of the polymer film would be measured with a Keithley Model 2100 6 ½ Digit Multimeter. During the measurement, a constant current would be applied and the voltage through the film was recorded, providing a resistance value via Ohm's law.

### Preparation of MIP-SWNT Suspensions

In another prophetic example, the MIP-coated nanotubes could be prepared by suspending 20 mg of SWNTs (BuckyUSA BU-202, 0.5-10 μm in length, 0.7-2.5 nm in diameter), 10 mg of PVPy (Polysciences, Inc. Cat#:01051 MW: 40,000), and a sufficient amount of a target molecule. A control suspension would then be produced with the identical mixture minus the target molecule. Both suspensions would be sonicated for a sufficient time to achieve complete mixing. After sonication, the suspensions would be filtered through a funnel containing a frit with 4.5-5 μm pores. The CNTs left on the frit would then be washed with an appropriate solvent in order to remove any unbound PVPy or target molecule. The dried, coated CNTs would then be re-suspended in 20 mL of solvent by sonication a sufficient amount of time to achieve a uniform suspension.

The suspension could then be cast or otherwise coated onto a substrate as part of a sensor for the target molecule.

Having described several embodiments, it will be recognized by those skilled in the art that various modifications, alternative constructions, and equivalents can be used without departing from the spirit of the disclosure. Accordingly, the above description should not be taken as limiting the scope of the disclosure.

Those skilled in the art will appreciate that the presently disclosed instrumentalities teach by way of example and not by limitation. Therefore, the matter contained in the above description or shown in the accompanying drawings should be interpreted as illustrative and not in a limiting sense. The following claims are intended to cover all generic and specific features described herein.

1. A sensor for the detection of a waterborne target molecule comprising a molecularly imprinted polymer film and a surface, said molecularly imprinted polymer film comprising a polymer host comprising binding sites for said waterborne target molecule, and
wherein said molecularly imprinted polymer film is coated upon said surface and said molecularly imprinted polymer film comprises a polymer host comprising a structural component and a conductive component.

2. (canceled)

3. The sensor of claim 1, wherein said structural component of the sensor comprises poly(4-vinylphenol), polyurethane, nylons, poly(2-vinylpyrole), poly(vinylpyridine) (PVP), poly(methylmethacrylate) (PMMA), acrylates, nylon-6, polyethyleneimine, polyurethane, polycarbonate, polyvinylpyrrolidinone and polystryrene.

4. The sensor of claim 1, wherein said conductive component comprises polyaniline, carbon nanotubes, and/or single wall carbon nanotubes.

5. The sensor of claim 1, wherein said waterborne target molecule is selected from the group consisting of chlorinated solvents, carbon tetrachloride, organophosphates, cyclic volatile methylsiloxane, endocrines, endocrine mimics, estrogens, organobromides, and decabromodiphenyl ether.

6. The sensor of claim 1, wherein said structural component, said conductive component and said target molecule are soluble in a first solvent selected from the group consisting of alcohols, dimethylformamide, and chloroform.

7. The sensor of claim 1, wherein said surface comprises an electrode.

8-9. (canceled)

10. The sensor of claim 1, wherein said molecularly imprinted polymer film has a thickness equal to or less than about 0.25 inches.

11. A method for detecting an waterborne target molecule using a molecularly imprinted polymer film or sensor of claim 1, said method comprising exposing said molecularly imprinted polymer film coated surface to a solution, and
measuring the resistance to the flow of an electrical current applied to said molecularly imprinted polymer film coated surface, and
wherein said resistance measurement is used to detect said waterborne target molecule in said solution.

12-13. (canceled)

14. A sensor for the detection of a waterborne target molecule comprising a molecularly imprinted polymer film and a surface, said molecularly imprinted polymer film comprising carbon nanotubes coated with a molecular imprinted polymer comprising binding sites for said waterborne target molecule, and
wherein said molecularly imprinted polymer film is coated upon said surface.

15. The sensor of claim 14, wherein said surface comprises an electrode.

16-21. (canceled)

22. A method for producing a molecularly imprinted polymer film for detection of a waterborne target molecule, said method comprising:
dissolving a polymer host comprising a structural component and a conductive component in a first solvent to form a first solution;
adding a target molecule to said first solution;
mixing said target molecule into said first solution to form a molecularly imprinted polymer solution;
coating said molecularly imprinted polymer solution onto a surface; and
removing said target molecule to form said molecularly imprinted polymer film.

23. The method of claim 22, said coating comprising electropolymerization, spin casting or laser deposition.

24. The method of claim 22, said step of removing said target molecule comprising:
extracting said target molecule from said molecularly imprinted polymer film using a second solvent,
wherein said polymer host is insoluble in said second solvent, and
wherein said target molecule is soluble in said second solvent.

25. The method of claim 22, wherein said first solvent has a boiling point lower than the boiling point of said target molecule.

26. The method of claim 25, said step of removing said target molecule comprising evaporating said target molecule from said molecularly imprinted polymer film.

27. The method of claim 22, wherein said target molecule is selected from the group consisting essentially of chlorinated solvents, carbon tetrachloride, organophosphates, cyclic volatile methylsiloxane, endocrines, endocrine mimics, estrogens, organobromides, and decabromodiphenyl ether.

28. The method of claim 27, wherein said target molecule comprises homologs of said waterborne target molecules.

29. The method of claim 27, wherein said first solvent is selected from the group consisting of alcohols, dimethylformamide, and chloroform.

30. The method of claim 22, wherein said first solvent is formic acid.

31. The method of claim 22, wherein said structural component of the sensor comprises poly(4-vinylphenol), polyurethane, nylons, poly(2-vinylpyrole), poly(vinylpyridine) (PVP), poly(methylmethacrylate) (PMMA), acrylates, nylon-6, polyethyleneimine, polyurethane, polycarbonate, polyvinylpyrrolidinone and polystryrene.

32. The method of claim 31, wherein said conductive component comprises polyaniline, carbon nanotubes, and/or single wall carbon nanotubes.

33. The method of claim 31, wherein said polymer host comprises nylon-6 and polyaniline.

34. The method of claim 31, wherein said polymer host comprises polyethyleneimine and polyaniline.

35. The method of claim 22, wherein said polymer host ranges from about 2 percent to about 15 percent by weight with respect to said first solvent in said first solution.

36. The method of claim 22, wherein said target molecule ranges from about 2 percent to about 10 percent by weight with respect to said first solvent in said molecularly imprinted polymer solution.

37-39. (canceled)

40. The method of claim 22, wherein said molecularly imprinted polymer film composition comprises a molar ratio of about 1 to 1 of said conductive component and said structural component.

41-43. (canceled)

44. A method for removing a waterborne target molecule from a solution comprising,
using a molecularly imprinted polymer film for the detection of a waterborne target molecule produced claim 22 in a chromatographic process,
wherein said solution is passed through said molecularly imprinted polymer film.

45. A solid phase extraction molecularly imprinted polymer comprising a polymer host comprising binding sites for a waterborne target molecule, said polymer host comprising a structural component and a conductive component.

46. (canceled)

47. The solid phase extraction molecularly imprinted polymer of claim 45, wherein said structural component comprises poly(4-vinylphenol), polyurethane, nylons, poly(2-vinylpyrole), poly(vinylpyridine) (PVP), poly(methylmethacrylate) (PMMA), acrylates, nylon-6, polyethyleneimine, polyurethane, polycarbonate, polyvinylpyrrolidinone and polystryrene.

48. The solid phase extraction molecularly imprinted polymer of claim 45, wherein said conductive component comprises polyaniline, carbon nanotubes, and/or single wall carbon nanotubes.

49. The solid phase extraction molecularly imprinted polymer of claim 45, wherein said polymer host comprises nylon-6 and polyaniline.

50. The solid phase extraction molecularly imprinted polymer of claim 45, wherein said polymer host comprises polyethyleneimine and polyaniline.

51. The solid phase extraction molecularly imprinted polymer of claim 45, comprising said polymer host from about 2 percent to about 15 percent by weight; and
from about 2 to about 10 percent by weight of said target molecule.

52. The solid phase extraction molecularly imprinted polymer of claim 45 comprising a molar ratio of about 1 to 1 of said conductive component and said structural component, and
wherein said target molecule is selected from the group consisting essentially of chlorinated solvents, carbon tetrachloride, organophosphates, cyclic volatile methylsiloxane, endocrines, endocrine mimics, estrogens, organobromides, and decabromodiphenyl ether.

53. The solid phase extraction molecularly imprinted polymer of claim 45, wherein said waterborne target molecule is selected from the group consisting of chlorinated solvents, carbon tetrachloride, organophosphates, cyclic volatile methylsiloxane, endocrines, endocrine mimics, estrogens, organobromides, and decabromodiphenyl ether.

54. The solid phase extraction molecularly imprinted polymer of claim 45, wherein the solid phase extraction molecularly imprinted polymer is in powder form.

55. A method for removing a waterborne target molecule from a solution comprising,
using a solid phase extraction molecularly imprinted polymer of claim 54 in a chromatographic process,
wherein a solution is passed through said solid phase extraction molecularly imprinted polymer.
